(19) United States
(12) Patent Application Publication
(10) Pub. No.: US 2005/0085445 A1
(43) Pub. Date: Apr. 21, 2005

(54) Cyclodextrines for use as suspension stabilizers in pressure-liquefied propellants

(76) Inventors: Bernd W Muller, Flintbek (DE); Hartwig Steckel, Schonkirchen (DE); Sebastian Wehle, Kiel (DE)

Correspondence Address: NIXON & VANDERHYE, PC, 1100 N GLEBE ROAD, 8TH FLOOR, ARLINGTON, VA, 22201-4714, US

(21) Appl. No.: 10/503,470

(22) PCT Filed: Feb. 6, 2003
(86) PCT No.: PCT/EP03/01180

(30) Foreign Application Priority Data
Feb. 7, 2002 (DE) 102 05 087.2

(51) Int. Cl. A61L 9/04; A61K 9/14; A61K 31/724

(52) U.S. Cl. 514/58; 424/46

(57) ABSTRACT

Metered-dose aerosol inhaler composition, which contains a) at least one pharmaceutical active ingredient, b) at least one propellant, c) at least one native or modified cyclodextrine, d) at least one hydrophilic additive, and optionally ethanol; process for the preparation of a metered-dose aerosol inhaler composition; as well as the use of a combination of cyclodextrine with a hydrophilic additive as suspension stabilizer in a metered-dose aerosol inhaler.

The present invention relates to a metered-dose aerosol inhaler, which contains at least one native or modified cyclodextrine, a process for producing the metered-dose aerosol inhaler and the use of cyclodextrines in a metered-dose aerosol inhaler.

Metered-dose aerosol inhalers are aerosol inhalers which, by means of a pressure-liquefied gas and a dosing valve, make possible the use by inhalation of active ingredients in humans or animals. Metered-dose aerosol inhalers combine the advantages of a high dosing accuracy, independence of the patient due to its portability as well as a high number of available doses. Compared with the oral administration of active ingredients, metered-dose aerosol inhalers also have less potential for side-effects.

At present there are two processes for avoiding the difficulties which arise during the formulation of a stable aerosol inhaler. The first production process consists of dissolving the active ingredients in the propellant with the aid of a co-solvent, forming a true solution. This process has been described e.g. by Steckel and Müller (H. Steckel and B. W. Müller, Metered-dose inhaler formulations with beclomethasone diproprionate using the ozone friendly propellant R 134a, Eur. J. Pharm. Biopharm. (1997) 77-83). By the addition of ethanol as co-solvent, the active ingredient, in this case the beclomethasone diproprionate, can be held in solution. As most active ingredients, even with the use of high ethanol concentrations, do not however dissolve in the modern propellants such as 2H-heptafluoropropane (HFA 227) and 1,1,1,2-tetrafluoroethane (HFA 134a), this process cannot be used universally.

The second process for producing a metered-dose aerosol inhaler consists of suspending the active ingredient in the propellant by the addition of adjuvants. For a high efficiency of the aerosol inhaler in the respiratory tracts, the particle size of the active ingredient components is of decisive importance. Particles which are smaller than 5 μm are regarded as lung-accessible. Particles which are smaller than 3 μm reach the deep sections of the lungs and thus rapidly get into the body's circulation or act locally. The production of a suspension metered-dose aerosol inhaler consequently includes the production and maintenance of a suitable particle-size distribution. The addition of adjuvants can stabilize the particles suspended in the propellant e.g. with regard to the particles' clumping, adhering to the wall of the transport container or a sedimentation or creaming.

Disadvantages of such a production process are the expensive adjustment of the particle-size distribution of the active ingredient components to an inhalable size and the avoidance of a change in the size distribution over a suitable storage period. In order to stabilize the crushed active ingredient sufficiently in the liquefied propellant, a surfactant is added which dissolves in the propellant. Up to now the surfactants oleic acid, lecithin and sorbitan trioleate have been used. These three surfactants, permitted for use by inhalation, are however not soluble in sufficient quantities in the modern propellants (2H-heptafluoropropane (HFA 227) and 1,1,1,2-tetrafluoroethane (HFA 134a), with the result that no suspension stabilization takes place. It must therefore be attempted, by using a co-solvent such as ethanol, to keep the surfactants in solution, in order to achieve a suspension-stabilizing effect, as disclosed e.g. in EP-B1-0 372 777. In a few cases, a stable suspension may also result without further addition of surfactants or other adjuvants, as disclosed by Steckel and Müller (H. Steckel and B. W. Müller, Metered-dose inhaler formulation of fluticasone 17-propionate micronized with super-critical carbon dioxide using the alternative propellant HFA-227, Int. J. Pharm. 173 (1998) 25-33) and in U.S. Pat. No. 5,891,420. Another possibility is to seek new surfactants which have better solubility in the propellants (DE-A-42 08 545 and U.S. Pat. No. 6,054,488). In the latter two documents for example the use of ethoxylated and non-ethoxylated partial glycerides as a suspension stabilizer is disclosed.

The named processes can however only be used for specific active ingredients and not universally. In addition, the change in propellants from CFCs which damage the ozone layer to ozone-friendly HFCs such as 2H-heptafluoropropane (HFA 227) and 1,1,1,2-tetrafluoroethane (HFA 134a) is leading to problems in the production of metered-dose aerosol inhalers due to the different physicochemical properties of the new propellants.

Therefore, the object of the invention was to make available a composition in which any pharmaceutical active ingredients are formulated with the modern propellants, in particular HFA 227 and HFA 134a, to produce stable suspension metered-dose aerosol inhalers.

It is known from EP-A-0 349 091 that, when dimethyl-β-cyclodextrine is used, pharmaceutical compositions which contain 17-β-oestradiol and/or progesterone, are suitable for nasal administration. The publication is however inter alia not concerned with the problems which occur with HFA propellants, but with aqueous solutions.

WO 90/15792 discloses inclusion complexes of cyclodextrines with specific active ingredients in aqueous solution for the treatment of heart diseases, but not metered-dose inhalers.

DE-A-31 18 218 discloses that, by methylation of β-cyclodextrine, the water-solubility of inclusion complexes of the cyclodextrines with biologically active organic substances is improved.

Preliminary tests for the present invention produced the following findings:
1. A combination of cyclodextrine with ethanol does not provide a stable suspension with propellant and active ingredient. After the addition of propellant to the mixture of ethanol, cyclodextrine and active ingredient, a milky suspension is produced, which within one day shows the typical disadvantages of a standard suspension. The particles clump together and float on the propellant. This clumping cannot be reversed even by intensive shaking. Use of the metered-dose inhaler is also made impossible by a potential failure of the dosing valve.
2. A combination of polyethylene glycol with ethanol does not provide a stable suspension with propellant and active ingredient. From the mixture of polyethylene glycol with ethanol and active ingredient, on addition of propellant what looks like a solvent is produced at first. After a week's storage the preparation shows a clear crystal growth of the previously dissolved medicament. Such formulations lose their therapeutic effectiveness by the removal of the medicament and therefore cannot be used for exact dosing.

In contrast, it has surprisingly been found that a composition according to the invention, containing
a) at least one pharmaceutical active ingredient,
b) at least one propellant,
c) at least one native or modified cyclodextrine and
d) at least one hydrophilic additive such as e.g. polyethylene glycol, polyvinylpyrrolidone, macrogol fatty acid ester, macrogol fatty acid ether or macrogol glycerol fatty acid ester,
   forms a suspension, which optically shows no sign of ageing over a prolonged storage period (longer than 6 months).

The suspension according to the invention shows no sign of separation of its components, the active ingredient does not float on the propellant (flotation) or clump together to form a cake on the bottom of the vessel containing the preparation. Metered-dose aerosol inhaler compositions according to the invention show a high inhalable fraction, with a high dosing accuracy of the formulation and complete functionality of the dosing valve. Adding the named components together results in spontaneous formation of a stable suspension.

A preferred composition according to the invention also contains e) ethanol. Further preferred versions are a subject of the dependent claims.

Examples of preferred pharmaceutical active ingredients used are anti-asthmatic agents such as budesonide, beclomethasone, dexamethasone, flunisolide, fluticasone, hydrocortisone, triamcindlone, adrenaline, bitolterol, clenbuterol, ephedrine, fenoterol, formoterol, isoproterenol, noradrenaline, pirbuterol, reproterol, salbutamol, salmeterol, terbutaline, ipratropium, oxitropium, tiotropium, nedocromil, cromoglycic acid, salts or esters of the abovementioned compounds or combinations of the named active ingredients; systemically active substances such as atropine, buprenorphine, fentanyl, morphine, glibenclamide, prednisone, prednisolone, scopolamine, sildenafil, apomorphine or their salts and derivatives; anti-infective agents such as e.g. tobramycin, gentamycin, ciclosporin; systemically active proteins, peptides, plasmids or DNA fragments such as e.g. insulin, α₁-antitrypsin, calcitonin, desmopressin, human growth hormone and other hormonally active substances such as systemic vaccines or immunoglobulines.

Examples of preferred propellants used are 2H-heptafluoropropane (HFA 227) and 1,1,1,2-tetrafluoroethane (HFA 134a) and mixtures thereof.

The cyclodextrine used according to the invention can be a native or modified α-, β-, or γ-cyclodextrine. Examples of modified cyclodextrines are hydroxymethyl-α-cyclodextrine, hydroxyethyl-α-cyclodextrine, hydroxypropyl-α-cyclodextrine, α-cyclodextrine butyl sulphonate, α-cyclodextrine butyl fluoride and sulphobutyl-α-cyclodextrine; hydroxymethyl-β-cyclodextrine, hydroxyethyl-β-cyclodextrine, hydroxypropyl-β-cyclodextrine, β-cyclodextrine butyl sulphonate, β-cyclodextrine butyl fluoride and sulphobutyl-α-cyclodextrine as well as hydroxymethyl-γ-cyclodextrine, hydroxyethyl-γ-cyclodextrine, hydroxypropyl-γ-cyclodextrine, γ-cyclodextrine butyl sulphonate, γ-cyclodextrine butyl fluoride and sulphobutyl-γ-cyclodextrine.

In a preferred version the total content of components c) and d) and optionally e) amounts to 0.01 to 30 wt.-%, preferably 0.1 to 20 wt.-%, in particular 0.5 to 15 wt. %, relative to the total mass of the preparation.

The weight ratio of component a) active ingredient(s) to component c) cyclodextrine(s) preferably lies within the range of 10:1 to 1:100, preferably 5:1 to 1:50, in particular 2:1 to 1:20. In preferred compositions according to the invention the propellant component b) amounts to 50 to 99 wt.-%, preferably 75 to 98 wt.-%, in particular 80 to 97 wt.-%, for example 90 to 95 wt.-%, relative to the total mass of the preparation.

Moreover a composition f) according to the invention can contain one or more conventionally used adjuvants. Such adjuvants as, e.g. lipophilic surfactants glycerol or propylene glycol are sufficiently known.

In addition the invention relates to a process for producing a metered-dose aerosol inhaler composition, in which a mixture which contains a) at least one pharmaceutical active ingredient, c) at least one native or modified cyclodextrine, d) at least one hydrophilic additive and optionally e) ethanol, is produced and the mixture is converted to a suspension by the addition of at least one propellant.

For example the pharmaceutical active ingredient is first converted to a real solution by mixing with ethanol, cyclodextrine and polyethylene glycol at room temperature. If the previously pressure-liquefied propellant (e.g. HFA 227 or 134a) or a mixture of the two propellants is added to this solution, a milky suspension according to the invention is spontaneously formed without the influence of external energy (stirring, dispersal, homogenization).

The dispersal of the adjuvants in a solution of the pharmaceutical active ingredient in ethanol to a fine dispersion also leads spontaneously, after the addition of a pressure-liquefied propellant, to a suspension of fine particles, which is stabilized by the adjuvants present.

The advantages of the invention are made clear by the following examples:

### EXAMPLE 1

36 mg of salbutamol are dissolved with 300 mg of polyethylene glycol 200, 600 mg of ethanol and 500 mg of hydroxypropyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 200 μg of salbutamol are released per stroke.

### EXAMPLE 2

36 mg of salbutamol are dissolved with 250 mg of polyethylene glycol 300, 400 mg of ethanol and 500 mg of hydroxymethyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 200 μg of salbutamol are released per stroke.

### EXAMPLE 3

36 mg of budesonide are dissolved with 200 mg of polyethylene glycol 300, 400 mg of ethanol and 160 mg of hydroxypropyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 200 μg of budesonide are released per stroke.

### EXAMPLE 4

1.8 mg of formoterol fumarate dihydrate are dissolved with 200 mg of polyethylene glycol 600, 400 mg of ethanol and 100 mg of hydroxypropyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 10 μg of formoterol fumarate dihydrate are released per stroke.

### EXAMPLE 5

36 mg of budesonide and 1.8 mg of formoterol fumarate dihydrate are dissolved with 200 mg of polyethylene glycol 300, 400 mg of ethanol and 200 mg of hydroxypropyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 200 μg of budesonide and 10 μg formoterol fumarate dihydrate are released per stroke.

### EXAMPLE 6

45 mg of fluticasone-17-propionate are dissolved with 300 mg of polyethylene glycol 300, 600 mg of ethanol and 300 mg of hydroxypropyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 250 μg of fluticasone-17-propionate are released per stroke.

### EXAMPLE 7

1.8 mg of formoterol fumarate dihydrate are dissolved with 200 mg of polyethylene glycol 600, 500 mg of ethanol and 20 mg of hydroxymethyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 10 μg of formoterol fumarate dihydrate are released per stroke.

### EXAMPLE 8

9 mg of salmeterol xinafoate are dissolved with 300 mg of polyethylene glycol 300, 600 mg of ethanol and 40 mg of hydroxypropyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 50 μg of salmeterol xinafoate are released per stroke.

### EXAMPLE 9

36 mg of budesonide are dissolved with 300 mg of polyethylene glycol 200, 600 mg of ethanol and 200 mg of hydroxypropyl-β-cyclodextrine at room temperature. This solution is poured into a pressure-resistant glass flask and sealed in with a dosing valve which releases 50 μl per stroke, then made up to 12.5 g with a mixture of propellants HFA 227 and HFA 134a in a ratio of 80 to 20 introduced through the valve. Thus approximately 200 μg of budesonide are released per stroke.

### EXAMPLE 10

10 mg of porcine insulin are dissolved with 300 mg of polyethylene glycol 200, 600 mg of ethanol and 100 mg of hydroxypropyl-β-cyclodextrine at room temperature. This solution is poured into a pressure-resistant glass flask and sealed in with a dosing valve which releases 50 μl per stroke, then made up to 12.5 g with propellant HFA 227 introduced through the valve. Thus approximately 55 μg of porcine insulin are released per stroke.

1. Metered-dose aerosol inhaler composition, which contains
a) at least one pharmaceutical active ingredient,
b) at least one propellant,
c) at least one native or modified cyclodextrine and
d) at least one hydrophilic additive.

2. Composition according to claim 1, characterized in that it also contains e) ethanol.

3. Composition according to claim 1, characterized in that the active ingredient is
(i) an anti-asthmatic agent in particular
budesonide, beclomethasone, dexamethasone, flunisolide, fluticasone, hydrocortisone, triamcinolone, adrenaline, bitolterol, clenbuterol, ephedrine, fenoterol, formoterol, isoproterenol, noradrenaline, pirbuterol, reproterol, salbutamol, salmeterol, terbutaline, ipratropium, oxitropium, tiotropium, nedocromil, cromoglycic acid or salts or esters of the abovementioned compounds or combinations thereof,
(ii) a systemically active substance in particular atropine, buprenorphine, fentanyl, morphine, glibenclamide, prednisone, prednisolone, scopolamine, sildenafil, apomorphine or their salts and derivatives as well as related substances,
(iii) an anti-infective agent in particular e.g. tobramycin, gentamycin, ciclosporin,
(iv) a systemically active protein, peptide, plasmid or DNA fragment, in particular insulin, α1-antitrypsin, calcitonin, dornase-α, desmopressin, human growth hormone and other hormonally active substances,
(v) a prostaglandin derivative, in particular alprosadil, prostaglandin E2 and other tissue hormones.
(vi) a systemically active vaccine or immunoglobin or
(vii) a hormonally active substance.

4. Composition according to claim 1, characterized in that the propellant is 2H-heptafluoropropane (HFA 227) and 1,1,1,2-tetrafluoroethane (HFA 134a) or a mixture of the two.

5. Composition according to claim 1, characterized in that the cyclodextrine is an α-, β- or γ-cyclodextrine.

6. Composition according to claim 1, characterized in that the modified α-, β- or γ-cyclodextrine is a hydroxymethyl cyclodextrine, hydroxyethyl cyclodextrine, hydroxypropyl cyclodex-trine, cyclodextrine butyl sulphonate, cyclodex-trine butyl fluoride and sulphobutyl cyclodextrine.

7. Composition according to claim 1, characterized in that the total content of components c) and d) and optionally e) amounts to 0.01 to 30 wt.-%, preferably 0.1 to 20 wt.-%, in particular 0.5 to 15 wt.-%, relative to the total mass of the preparation.

8. Composition according to claim 1, characterized in that the weight ratio of component a) active ingredient(s) to component c) cyclodextrine(s) lies within the range of 10:1 to 1:100, preferably 5:1 to 1:50, in particular 2:1 to 1:20.

9. Composition according to claim 1, characterized in that the propellant component b) amounts to 50 to 99 wt.-%, preferably 75 to 98 wt.-%, in particular 80 to 97 wt.-%, for example 90 to 95 wt.-%, relative to the total mass of the preparation.

10. Composition according to claim 1, characterized in that the hydrophilic additive is selected from the group of polyethylene glycols with an average molecular weight of 100 to 30,000, preferably with an average molecular weight of 200 to 6000 and preferably with a molecular weight of 200 to 1000.

11. Composition according to claim 1, characterized in that the hydrophilic additive is a mixture of polyethylene glycols with low and high molecular weight.

12. Composition according to claim 1, characterized in that the hydrophilic additive is selected from the group of polyethylene glycol fatty acid ethers (Brij substances), the group of polyethylene glycol fatty acid esters (Myrj substances), the group of polyethylene glycol glycerol fatty acid esters (e.g. Tagat S, Tagat O, Solutol HS 15, Cremophor EL, Cremophor S9), the group of polysorbates or the group of polyoxyethylene-polypropylene block copolymers, e.g. Poloxamer 188, Synperonic P/E L 61, Pluronic F 68.

13. Composition according to claim 1, characterized in that the hydrophilic additive has a polyethylene glycol proportion of 2 or more ethylene oxide units.

14. Composition according to claim 1, characterized in that the hydrophilic additive is a hydrophilic polymer, in particular polyvinylpyrrolidone, polyvinyl alcohol, cross-linked polyvinylpyrrolidone, vinylpyrrolidone-vinyl acetate-copolymer.

15. Process for producing a metered-dose inhaler aerosol composition in which a mixture which contains a) at least one pharmaceutical active ingredient, c) at least one native or modified cyclodextrine, d) at least one hydrophilic additive and optionally e) ethanol is produced, and, by the addition of b) at least one propellant, the mixture is converted to a stable, non-sedimenting suspension.

16. Use of a combination of cyclodextrine with a hydrophilic additive as suspension stabilizer in a propellant-containing metered-dose aerosol inhaler, which contains at least one pharmaceutical active ingredient.
